(19) United States
(12) Patent Application Publication
(10) Pub. No.: US 2026/0154813 A1
(43) Pub. Date: Jun. 4, 2026

(54) PLATFORM BASED PREDICTIONS USING DIGITAL PATHOLOGY INFORMATION

(71) Applicant: Verily Life Sciences LLC, Dallas, TX (US)

(72) Inventors: Yang Wang, Mountain View, CA (US); Niranjan Sridhar, South San Francisco, CA (US); Carson McNeil, San Bruno, CA (US); Andrew Homyk, South San Francisco, CA (US); Cheng-Hsun Wu, South San Francisco, CA (US); Ali Behrooz, South San Francisco, CA (US)

(21) Appl. No.: 19/123,558

(22) PCT Filed: Oct. 2, 2023
(86) PCT No.: PCT/US2023/075723

§ 371 Date: Apr. 23, 2025

Related U.S. Application Data
(60) Provisional application No. 63/421,046, filed on Oct. 31, 2022.

(51) Int. Cl. G06T 7/00; G06T 11/10; G06V 10/26; G06V 10/764; G06V 20/70; G16H 50/20

(52) U.S. Cl. CPC G06T 7/0012; G06T 11/10; G16H 50/20; G06T 2207/10024; G06T 2207/10056; G06T 2207/10064; G06T 2207/20072; G06T 2207/20081; G06T 2207/20092; G06T 2207/30024; G06T 2207/30056; G06V 10/26; G06V 10/764; G06V 20/70; G06V 2201/031

(57) ABSTRACT

A computer system may enable an end-to-end platform for evaluating digital pathology information. An example process that uses the platform may include receiving first image data corresponding to a tissue sample. The process may also include generating second image data from the first image data by applying at least one virtual stain to the first image data, where at least one virtual stain is selected based on a target clinical diagnosis. The process may also include generating, by a predictive modeling suite, third image data from the second image data by identifying a plurality of histologic features present in the second image data in accordance with the target clinical diagnosis. The process may also include generating, by the predictive modeling suite and using the third image data, a clinical prediction relating to the target clinical diagnosis. The process may also include providing information associated with the clinical prediction for presentation.

### CROSS-REFERENCES TO RELATED APPLICATIONS

This international application claims priority to U.S. patent application Ser. No. 63/421,046, filed on Oct. 31, 2023, the disclosure of which is herein incorporated by reference in its entirety for all purposes.

### BACKGROUND

Recent advances in imaging technology and computer vision have enabled pathologists to use computers to assist in the evaluation of pathology slides. Conventionally, however, once imaging has been performed even using new approaches, the imaging still has to be evaluated by a trained pathologist using bright field imaging microscopes, which can be slow and prone to subjective results. Computer vision techniques have been shown to accelerate or improve diagnostic scoring, but these techniques have conventionally relied upon inefficient and industry-standard approaches for staining tissue samples.

### BRIEF SUMMARY

Various examples are described including systems, methods, and devices relating to an end-to-end digital pathology platform.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a computer-implemented method. The h method includes receiving first image data corresponding to a tissue sample, where the first image data represents an autofluorescence image. The computer-implemented method also includes generating second image data from the first image data by applying at least one virtual stain to the first image data, where the at least one virtual stain is selected based on a target clinical diagnosis. The computer-implemented method also includes generating, by a predictive modeling suite, third image data from the second image data by identifying a plurality of histologic features present in the second image data in accordance with the target clinical diagnosis. The computer-implemented method also includes generating, by the predictive modeling suite and using the third image data, a clinical prediction relating to the target clinical diagnosis. The computer-implemented method also includes providing information associated with the clinical prediction for presentation. Other embodiments of this aspect include corresponding computer systems, apparatus, and computer programs recorded on one or more computer storage devices, each configured to perform the actions of the methods.

One general aspect includes a system. The system also includes a microscope system configured to capture a first set of images of a tissue sample. The system also includes a virtual stainer model configured to: receive the first set of images from the microscope system; and convert the first set of images into a second set of images based at least in part on color information associated with a target clinical diagnosis, where each image in the second set of images includes a virtual stain. The system also includes a stage one predictive model configured to: receive, as input, the second set of images; and output a third set of images by converting the second set of images into the third set of images based at least in part on histologic feature information associated with the target clinical diagnosis, where each image in the third set of images includes one or more histologic annotations corresponding to the target clinical diagnosis. The system also includes a stage two predictive model configured to: receive, as input, the third set of images; and output a prediction corresponding to the target clinical diagnosis based at least in part on the histologic feature information. Other embodiments of this aspect include corresponding methods, apparatus, and computer programs recorded on one or more computer storage devices, each configured to perform the actions described with respect to the system.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 illustrates a block diagram illustrating a process for platform-based evaluation of digital pathology information, according to at least one example.

FIG. 2 illustrates an example system for platform-based evaluation of digital pathology information, according to at least one example.

FIG. 3 illustrates an example virtual stainer model for platform-based evaluation of digital pathology information, according to at least one example.

FIG. 4 illustrates an example block diagram illustrating an aspect of platform-based evaluation of digital pathology information, according to at least one example.

FIG. 5 illustrates an example of a flow chart depicting an example process for evaluating digital pathology information using an end-to-end digital pathology platform, according to at least one example.

FIG. 6 illustrates an example system for implementing techniques relating to platform-based evaluation of digital pathology information, according to at least one example.

### DETAILED DESCRIPTION

Examples are described herein in the context of a digital pathology and evaluation of a particular disease diagnosed using pathology (i.e., nonalcoholic steatohepatitis (NASH)). Those of ordinary skill in the art will realize that the following description is illustrative only and is not intended to be in any way limiting. For example, the techniques described herein can be used to evaluate tissue samples in connection with other tests for any others disease typically diagnosed from pathology samples. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application and business-related constraints, and that these specific goals will vary from one implementation to another and from one developer to another.

Conventional histopathology is used for the diagnosis of many diseases. This typically involves the extraction of tissues from subjects, sectioning out thin slices, followed by a manual process of staining, characterization, and scoring done by pathologists. However, the process is slow, time-consuming, expensive, and does not support tissue preservation for advanced molecular analysis of the sample. Described herein is an end-to-end workflow for pathology powered by hyperspectral microscopy and deep learning which addresses many of the key bottlenecks, inefficiencies, and inconsistencies in pathology today.

Conventional processes of staining tissues and imaging them are laborious and expensive. The requirement of chemical reagents and degrading effects of age and light on tissues impose additional costs. The approaches described herein address this problem by relying upon hyperspectral microscopy to non-destructively image tissue autofluorescence of unstained tissue sections, greatly increasing the information that can be collected.

Conventional staining and imaging processes require multiple irreversible transformations to be applied on the tissue. Therefore, if multiple assessments need to be done on a patient, the process has to be repeated from scratch on new tissue samples. The approaches descried herein address this problem by providing a deep learning based virtual histological staining technique, which can replace the cost of custom staining procedures and greatly extend the life and utility of tissue samples. The virtually stained images described herein may include suitable information for pathologists to identify all the salient tissue characteristics as if the images were real stained samples.

Conventional approaches for manually annotating and diagnosing diseases are complex, time-consuming, and require trained experts which can be expensive. Additionally, since the conventional approaches for staining and imaging are physical and chemical transformations performed by humans, they are susceptible to human-introduced variability. This can lead to considerable variability between staining, scoring, and diagnosis performed at different sites or different pathologists. The approaches described herein address these problems by providing a predictive modeling suite that uses deep learning segmentation models and Bayesian estimation for automated score prediction as it relates to a specific disease. These approaches have been shown to be at least as accurate, and more accurate in some cases, than human users.

With this backdrop, the approaches described herein include an end-to-end digital pathology platform for tissue-based diagnostics that uses tissue sections and performs imaging, virtual staining, and diagnosis in an efficient and reproducible workflow. At the end of the workflow, and at various points throughout the workflow, the tissue samples are still unaltered and available for other analyses. This means that information can be output to a pathologist at any stage of the workflow. The pathologist can modify the digital information, make comments, and provide feedback that can be ingested by the system and used to improve and/or otherwise adjust future predictions. The first step in the workflow includes the autofluorescence imaging of unstained tissue using a hyperspectral imaging microscope which offers multiple operational efficiencies over conventional imaging. The second step of the digital pathology platform is a deep learning-based virtual stainer model. The final step of the digital pathology platform is a suite of algorithmic methods to take the virtual stained images as input and return clinical diagnoses. The benefits, including diagnostic efficacy, of the digital pathology platform are described with reference to nonalcoholic steatohepatitis (NASH), which is a progressive form of nonalcoholic fatty liver disease (NAFLD) caused by a buildup of fat in the liver. These benefits are applicable to the diagnosis of other diseases as well.

This illustrative examples above are given to introduce the reader to the general subject matter discussed herein and the disclosure is not limited to this example. The following sections describe various additional non-limiting examples of end-to-end digital pathology platforms and techniques for generating clinical diagnosis using such platforms.

The techniques described herein provide one or more technical improvements to systems that implement digital pathology systems. These improvements relate to those already described herein (e.g., diagnostic efficacy, reproducibility, tissue conservation, and improved identification of certain pathologies), along with certain other improvements. For example, a predictive model described herein may be used to output results relating to analysis of a pathology sample, these results may be input back into the model for retraining to improve later outputs.

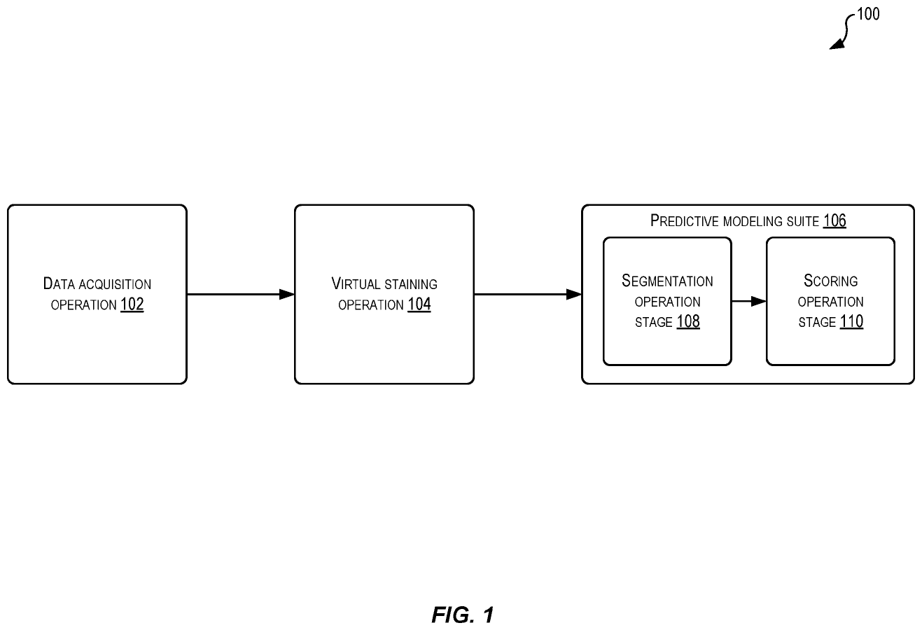

Turning now to the figures, FIG. 1 illustrates a block diagram illustrating a process 100 for platform-based evaluation of digital pathology information, according to at least one example. The process 100 may correspond to a high-level introduction to the digital pathology workflow described herein. Additional figures describe additional aspects of the workflow in more detail. In some examples, the workflow represented by the process 100 may be implemented under the control of a computer system, such as a desktop or laptop computer, server, cloud compute instance, and the like.

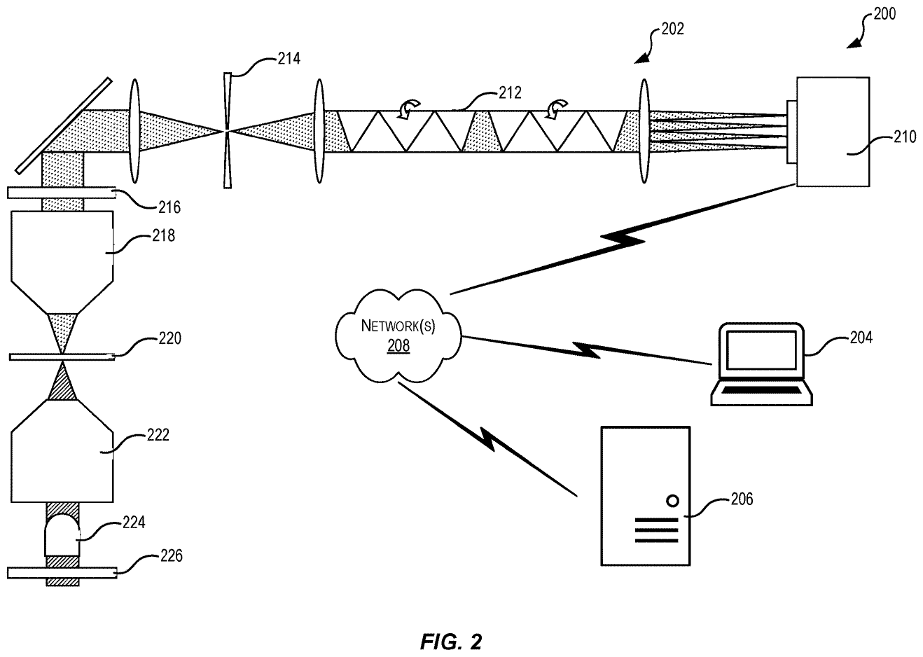

The process 100 may begin at block 102 with a data acquisition operation. The data acquisition operation 102 may include capturing raw images of a tissue sample, which can include a single image with multiple color channels or multiple different images each with a single color channel. The raw images may be autofluorescence images in order to highlight different properties of the tissue sample. The tissue sample may have been obtained, processed, and placed upon a slide by a pathologist or other human user using conventional techniques. A system to perform the data acquisition operation 102 is shown and described in further detail with respect to FIG. 2.

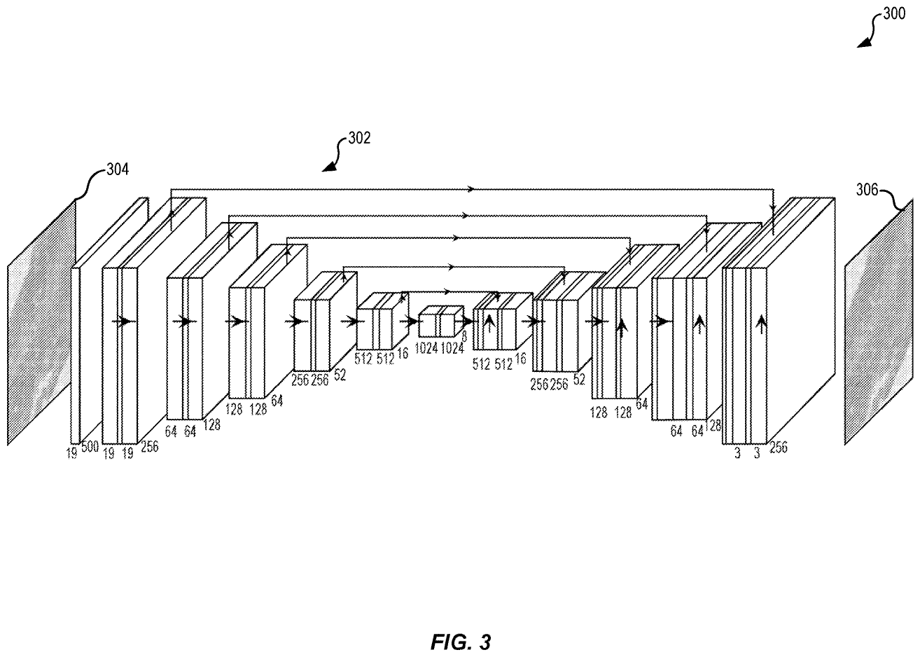

At the block identified by a virtual staining operation 104, the process 100 includes performing a virtual staining operation. The virtual staining operation 104 may include taking the images obtained at the block identified by 102 and loading them into a virtual staining model that applies virtual stains to the images to emulate real stains. The virtual staining model, as described in more detail in FIG. 3, may include any suitably-trained model that can apply virtual stains to raw images of tissue. The virtual staining operation of block 104 may include applying any suitable virtual stain. For the NASH example described herein, the stains may include trichrome and hematoxylin and eosin (H&E). Other stains may include, for example, Van Gieson, Toluidine blue, Alcian blue, Giemsa, Reticulin, Nissl, Orcein, Sudan black B, Masson's trichrome, Mallory's trichrome, Azan trichrome, Cason's trichrome, Periodic acid-Schiff, Weigert's resorcin-fuchsin, Wright and Wright-Giemsa, Aldehyde fuchsin, and any other suitable stain.

The next two blocks 108 and 110 of the process 100 may correspond to two modeling stages performed as part of a predictive modeling suite 106. Generally, the predictive modeling suite 106 may take the virtually stained images from block 104 and output a diagnosis relating to a particular disease (or at least a prediction with respect to the diagnosis). Thus, the predictive modeling suite 106 may be configured for a specific disease. As an example, a segmentation operation stage 108 may include taking virtually stained images from block 104 and identifying a set of biomarkers present in the image. These set of biomarkers may correspond to the disease under evaluation (e.g., NASH), and the segmentation operation stage 108 may mark up (or otherwise annotate) the virtually stained images to highlight biomarkers in the tissue sample that are uniquely associated with a NASH diagnosis. This can include annotating and counting cells, clusters of cells, types of cells, and the like.

At block 110, the process 100 includes the scoring operation stage of the predictive modeling suite 106. The scoring operation stage 110 may include taking the annotations from the segmentation operation stage 108 and applying scoring criteria to the features identified by the annotations. The scoring criteria may be specific to the disease under investigation. Example criteria may include a percentage of the image (e.g., sample) that has some property, a quantity of cells that include property, a proportion of cells having a first property compared to those having a second property, and the like. The scoring operation stage 110 may be quantitative, e.g., comparing a number of ballooning cells as compared to all cells in the tissue sample. The quantitative values may be compared to any suitable curve for evaluation, depending on the particular requirements of the scoring criteria. In some examples, the scoring operation stage 110 may apply bright line thresholds to the annotated images, again depending on the particular requirements of the scoring criteria.

FIG. 2 illustrates an example system 200 for platform-based evaluation of digital pathology information, according to at least one example. The system 200 may include a hyperspectral microscope 202, a local computer system 204, a remote computer system 206, and a network 208 to enable network communications between the hyperspectral microscope 202, the local computer system 204, and the remote computer system 206. The local computer system 204 may be any suitable computer system including, for example, a desktop computer, a laptop computer, a thin-client device, a tablet, and any other suitable device. The remote computer system 206 may also be any suitable computing device, which may include, for example, a cloud computing instance, a remote server, or any other server-based resource. The remote computer system 206 may be shared among customers or may be specific to a single customer.

The network 208 may be any suitable network to enable communications between the various elements of the system 200. In some examples, the network 208 may include more than one network. For example, the hyperspectral microscope 202 and the local computer system 204 may communicate via a first network and the local computer system 204 and the remote computer system 206 may communicate via a second network.

In some examples, different portions of the process 100 may be performed by elements of the system 200. For example, the hyperspectral microscope 202 may include its own onboard computing resources or may be operated under the control of the local computer system 204, which may be a standalone computer, or may be operated under the control of the remote computer system 206. In some examples, operation 102 may be performed by the hyperspectral microscope 202 and the local computer 204 and operations 104-110 may be performed by the remote computer system 206.

Turning now to the details of the hyperspectral microscope 202, which includes a camera 210, rotatable Janssen prisms 212, a confocal slit 214, a longpass filter 216, an emission objective 218, a sample 220 (e.g., a tissue sample), an excitation objective 222, a Powell lens 224, and a bandpass filter 226. Generally, the hyperspectral microscope 202 may be configured to capture images of the sample 220 having various autofluorescence values.

The hyperspectral microscope 202 may be a custom-built fluorescence microscope. The hyperspectral microscope 202 functions by focusing a transillumination excitation beam onto a line on the sample 220. The sample 220 is scanned perpendicular to the excitation-line axis to capture a single-traverse image across the sample 220, and this is repeated until the entire sample is covered. For each position along these traverses, fluorescence from the excited line is collected and imaged onto the coaligned confocal slit 214. A secondary relay incorporating dispersion is used to map the image onto a two-dimensional sensor, with one axis representing spatial information, and the perpendicular axis representing spectral information. This process may be repeated any suitable number of times. In a particular use case, five lasers may be used with wavelengths 355 nm, 405 nm, 488 nm, 561 nm, and 640 nm, each of which may be captured at multiple different emission frequencies. For example, five different light frequencies may include capturing 242 different emissions frequencies. Thus, the autofluorescence image may include 242 channels in at least one example.

Following scanning, as described above, the raw scan traverses are combined into a single hyperspectral image. This image is referred to herein as the autofluorescence image.

The image processing may include dark-frame subtraction, flat-field correction, and a spatial-spectral transformation. The profile used for the flat-field correction may first be estimated for each camera pixel as the median across all frames containing tissue. Each column of the flat-field profile may then be normalized to a mean of one. The spatial-spectral transformation may be a single trilinear interpolation from the camera frames to the final image. This may include corrections for a spectral shift (from the slit-camera alignment), lateral chromatic aberration, and registration errors between traverses. In regions where multiple traverses overlap, the mean intensity is used.

FIG. 3 illustrates an example virtual stainer model 300 for platform-based evaluation of digital pathology information, according to at least one example. Generally, the virtual stainer model may be a deep learning model inspired by the pix-to-pix translation models and adversarial networks. The model may include a main translation model 302 based on the well-known UNet pix-to-pix translation model (UNet) and a discriminator complex which may include three discriminators based on the Inception architecture which operate at three different scales. The virtual stainer model 300 may be configured to predict the appropriate virtual stains based on input parameters provided by an operator, properties of the autofluorescence images, and/or the models may be specifically configured for a certain virtual stain.

In some examples, the raw autofluorescence image output by the system 200 may be input into the virtual stainer model 300. As part of doing so, a predetermined set of 19 channels out of the 242 imaged by the system 200 may be selected. Next the pair-aligned AF and BF stained gigapixel images may be cut up into pairs of patches of size 256×256 pixels, thus the input to the virtual stainer model 300 is an image of shape 256×256×19 while the expected output is 256×256×3.

The input to the main translation model 302 is a patch of autofluorescence image 304, which may be of size 256×256×19. The main translation model 302 uses five convolutional blocks in both encoder and decoder. Each convolutional block is composed of a convolution, batch normalization, dropout, followed by a convolution and a batch normalization. In the encoder, each convolutional block is followed by a downsampling 1×1 convolution with stride 2 to reduce the size of the feature map while doubling the number of channels. The dimension of the embedding created after the encoder is 8×8×1024. In the decoder, each convolutional block is preceded by a bilinear upsampling and channel mixing convolutional layer. The final output of the main translation model 302 is a predicted virtually stained output image patch 306 having the predicted virtual stain of dimensions 256×256×3, where the three channels correspond to red, green, and blue (RGB).

A multiscale discriminator network is also used following the main translation model 302. The discriminator network may include three identical discriminator networks that take the input scaled to 0.5×, 1.0× and 2.0× of the original input. The discriminator takes both the original input autofluorescence image 304 and the predicted virtually stained output image patch 306, i.e, the input and output of the main translation model 302.

The additional patches of the input image may be processed using the virtual stainer model 300, and various virtual stains may be predicted using the virtual stainer model 300 or a different version of the model.

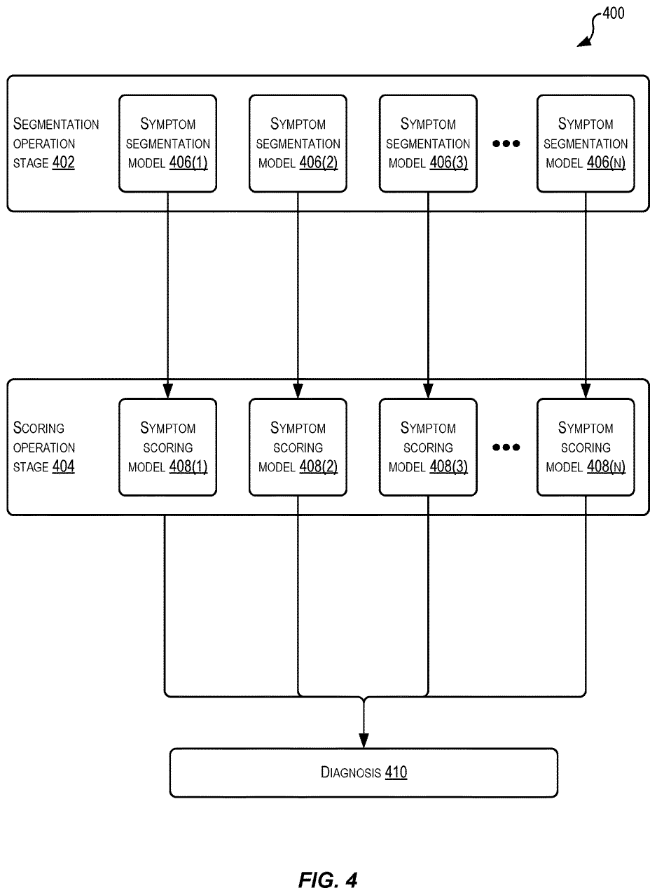

FIG. 4 illustrates an example block diagram 400 illustrating an aspect of platform-based evaluation of digital pathology information, according to at least one example. The diagram 400 includes a segmentation operation stage 402, a scoring operation stage 404, and a diagnosis 410. The segmentation operation stage 402 and the scoring operation stage 404 respectively correspond to the segmentation operation stage 108 and the scoring operation stage 110 described herein. Thus, the segmentation operation stage 402 and the scoring operation stage 404 may be different stages of the predictive modeling suite 106 described herein.

Generally, the segmentation operation stage 402 may take as input virtually stained images output by the virtual stainer model 300. The segmentation operation stage 402 includes a plurality of symptom segmentation models 406(1)-406(n). Each symptom segmentation model 406 may be trained to identify one or more image features that are used to diagnose a particular symptom of a specific disease. In the NASH example, there may be nine features that are used to diagnose four symptoms (e.g., lobular inflammation, steatosis, fibrosis, and ballooning). Thus, in the NASH example, the segmentation operation stage 402 may include four symptom segmentation models 406, each of which being trained to identify one of the four NASH symptoms. Output from each symptom segmentation model 406 may include annotations relevant to the specific symptom for which the model 406 has been trained. In other words, does the input patch include the required features? If so, annotate.

To train the symptom segmentation models 406, a panel of pathologists manually annotated a number of whole slide images of real tissues stained with H&E. The annotations captured the nine features that are used to diagnose the four symptoms of NASH CRN lobular inflammation, steatosis, fibrosis, and ballooning. The segmentation models were trained to use a stained image as input and predict the annotations as labels. This included segmenting whole slide images into small non-overlapping patches of 8×8 μm at 10× magnification and trained a convolutional neural network to classify each patch. The model architecture was based on Inception V3 [Inception] training on regions of 512×512 μm (512×512 pixels) to provide additional context for classifying the central 8×8 μm patch. Four different models were trained for each NASH symptom using annotations corresponding to each symptom. The output of each symptom segmentation model 406 is a binary probability score for every 8 μm×8 μm patch. Patchwise softmax cross-entropy has been used as the loss function to identify the optimal model hyperparameters using cross-validation. Regularization (via dropout, weight decay, and color perturbations) may also be beneficial. At inference, all the patches are joined back to produce a heatmap of feature predictions for the entire whole slide image, which is used to validate the model, visualize its outputs, and as input for the downstream scoring operation stage 404 and corresponding models. The training of the symptom segmentation models 406 essentially turned a classification task into a segmentation task.

The scoring operation stage 404 may include a plurality of symptom scoring models 408(1)-408(n). Generally, each symptom scoring model 408 of the scoring operation stage 404 may take as input the annotated images with feature predictions that are specific to each symptom. The function of each symptom scoring model 408 may be specific to the type of symptom at issue. For example, some models may sum up annotations present in the annotated images, others may compare area occupied by annotated regions compared to other areas. Described below are particular examples for the NASH-CRN segmentation and scoring models.

For the steatosis symptom, a steatosis segmentation model may predict a probability score between 0 and 1 for each 8 μm×8 μm patch. The symptom scoring model may then be used to calculate the steatosis fraction for the whole slide as the fraction of patches of the slide with a probability score above the threshold (e.g., 0.8). Finally, univariate Bayesian estimation may be used using the whole slide steatosis fraction to predict the clinical NASH-CRN steatosis score.

For the lobular inflammation symptom, the lobular inflammation segmentation model may predict a probability score between 0 and 1 for each 8 μm×8 μm patch. The symptom scoring model may then be used to calculate the lobular inflammation fraction of the whole slide as the fraction of patches of the slide with a probability score above the threshold (e.g., 0.7). Finally, univariate Bayesian estimation using the whole slide lobular inflammation fraction may be used to predict the clinical NASH-CRN lobular inflammation score.

For the ballooning symptom, five segmentation models were trained with different random initiations, each of which predicts a probability score between 0 and 1 for each 8 μm×8 μm patch. The symptom scoring model may then take the geometric mean of all the probability scores to get one probability score for each patch. Next, distinct connected patches may be labeled with probability scores greater than 0.5. Connected regions smaller than 4 patches and larger than 1000 patches may be discarded as anomalies. A single feature may be extracted—the log normalized ballooning count—by dividing the number of connected patch regions by the total tissue size and taking the logarithm of this number. Finally, univariate Bayesian estimation may be used using the whole slide steatosis fraction to predict the clinical NASH-CRN ballooning score.

For the fibrosis symptom, the fibrosis segmentation model assigns each 8 μm×8 μm patch into 1 of 5 classes: none, portal fibrosis, sinusoidal fibrosis, bridging fibrosis, or cirrhosis. Nine features may be calculated from this prediction map-pixel count, pixel density and component count for portal, sinusoidal and bridging fibrosis. Pixel count is simply the number of patches predicted for the respective class. Pixel density is pixel count divided by the number of patches in the whole slide, and component count is the number of connected component regions of the class. Finally, the symptom scoring model may include a random forest model that uses the nine features as input to predict the clinical fibrosis score.

The outputs from each symptom scoring model 408 may be combined in any suitable fashion to arrive at the diagnosis 410. For example, once the scores are known, a conventional scoring rubric for the particular disease may be referenced to determine the diagnosis. In some examples, the diagnosis 410 may be represented by a likelihood of the disease being present.

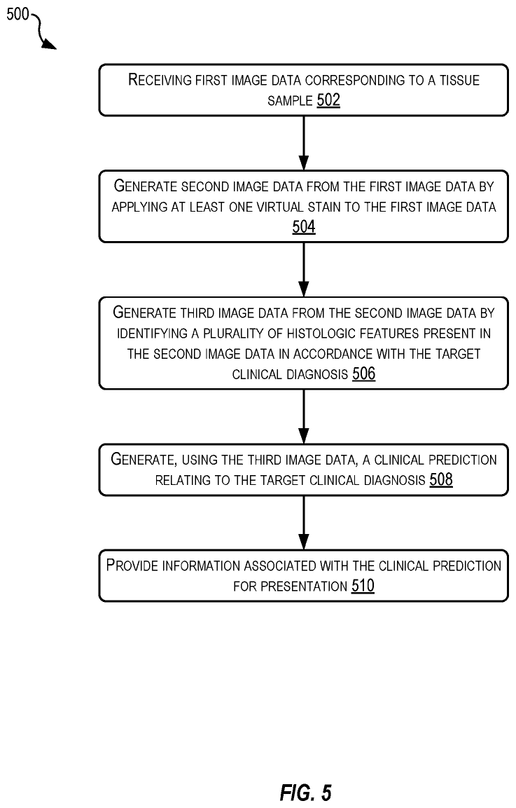

FIG. 5 illustrates an example flow diagram showing a process 500, according to at least a few examples. This process, and any other processes described herein (e.g., the process 100), are illustrated as logical flow diagrams, each operation of which represents a sequence of operations that can be implemented in hardware, computer instructions, or a combination thereof. In the context of computer instructions, the operations may represent computer-executable instructions stored on one or more non-transitory computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

Additionally, some, any, or all of the processes described herein may be performed under the control of one or more computer systems configured with specific executable instructions and may be implemented as code (e.g., executable instructions, one or more computer programs, or one or more applications) executing collectively on one or more processors, by hardware, or combinations thereof. As noted above, the code may be stored on a non-transitory computer-readable storage medium, for example, in the form of a computer program including a plurality of instructions executable by one or more processors.

In particular, FIG. 5 illustrates an example of a flow chart depicting an example process 500 for evaluating digital pathology information using an end-to-end digital pathology platform, according to at least one example. The process 500 is performed by the local computer system 204 (FIG. 2) and/or the remote computer system 206 (FIG. 2), which will be referred to herein generally as the computer system.

The process 500 begins at block 502 by the computer system receiving first image data corresponding to a tissue sample. The first image data may represent an autofluorescence image. In some examples, the first image data may represent a plurality of color channels. The first image data may be one or more images captured by the hyperspectral microscope 202.

In some examples, the process 500 may further include, prior to block 502, causing a microscope system to capture the first image data. The microscope system may include the hyperspectral microscope. The microscope may capture the first image data using a plurality of different light frequencies and a plurality of different emission frequencies to produce the plurality of color channels. For example, the image may be 256×256×9, or any other suitable size having any suitable number of channels.

At block 504, the process 500 includes the computer system generating second image data from the first image data by applying at least one virtual stain to the first image data. At least one virtual stain may be selected based on a target clinical diagnosis, corresponding to a target disease. The target disease may be one that is identifiable, at least in part, using tissue samples. In some examples, the second image data corresponds to a second image that is human-interpretable. The second image may be human-interpretable in the sense that a human such as a trained pathologist could look at the second image and decipher meaning from the image. In particular, the second image may be a virtual stained image of the tissue sample that corresponds to a real stain. In some examples, the process 500 may further include outputting the second image for review, approval, and/or editing by a human user. This may include presenting the second image at a display of the computer system and providing a user interface for the human user to interact with the second image.

As described herein, the target clinical diagnosis may include any suitable disease or other condition that may be diagnosed using pathology methods. The example described throughout this specification is NASH, though, others may also be evaluated using the techniques described herein. In some examples, more than one virtual stain may be selected and may therefore be used to create the second image data.

In some examples, generating the second image data from the first image data by applying at least one virtual stain to the first image data may include using the virtual stainer model 300, as described herein. In some examples, second image data may be output as patches, which are then combined again to create the second image.

At block 506, the process 500 includes the computer system generating third image data from the second image data by identifying a plurality of histologic features present in the second image data in accordance with the target clinical diagnosis. Generating the third image data may be performed by a predictive modeling suite.

In some examples, the plurality of histologic features may correspond to the target clinical diagnosis. For example, for a certain disease associated with the target clinical diagnosis, a set of histologic features (e.g., biomarkers) may be present in the second image data, and the block 506 may annotate the second image data with annotations to define the third image data. Thus, the third image data may include one or more images that include annotations that identify relevant features. As part of block 506, the process 500 may also include generating scores for a plurality of symptoms associated with the target clinical diagnosis based on the histologic features.

In some examples, the third image data corresponds to a set of heatmaps. In this example, each heatmap of the set of heatmaps represents a histologic feature of the plurality of histologic features. In some examples, the process 500 may further include outputting the set of heatmaps as a set of annotation overlays, wherein each annotation overlay of the set of annotation overlays is selectively viewable with respect to the second image. For example, the set of annotation overlays may be human-interpretable in the sense described herein. In some examples, a human user may provide input, comment on, and/or otherwise adjust the overlays. In some examples, each heatmap may correspond to a symptom of a plurality of symptoms associated with a particular disease.

At block 508, the process 500 includes the computer system generating, using the third image data, a clinical prediction relating to the target clinical diagnosis. Generating the clinical prediction relating to the target clinical diagnosis may be performed by the predictive modeling suite. In some examples, blocks 506 and 508 may be performed by the segmentation operation stage 402 and the scoring operation stage 404, and the clinical diagnosis may correspond to the diagnosis 410.

In some examples, generating the third image data from the second image data may include generating the third image data by a first stage of the predictive modeling suite. In this example, generating the clinical prediction may include generating the clinical prediction by a second stage of the predictive modeling suite.

In some examples, the first stage of the predictive modeling suite may include a plurality of predictive models, and the second stage of the predictive modeling suite may include a plurality of scoring models corresponding to the plurality of predictive models. In this example, the target clinical diagnosis may be associated with a plurality of diagnosis symptoms, and each predictive model of the plurality of predictive models and each scoring model of the plurality of scoring models may be configured according to a diagnosis symptom of the plurality of diagnosis symptoms.

In some examples, the target clinical diagnosis may be associated with a plurality of diagnosis symptoms. In this example, generating the clinical prediction may include generating a clinical score for each diagnosis symptom of the plurality of diagnosis symptoms based on a respective histologic feature of the plurality of histologic features, generating a composite score for the target clinical diagnosis based on the clinical score of each respective diagnosis symptom, and generating the clinical prediction using the composite score. In some examples, each diagnosis symptom of the plurality of diagnosis symptoms may be associated with a diagnostic rule set. In this example, the clinical prediction may be generated in accordance with the diagnostic rule set.

In some examples, the third image data may correspond to annotations of each histologic feature of the plurality of histologic features. In this example, the third image data may be associated with a region of a second image represented by the second image data. In this example, generating the clinical prediction may include generating the clinical prediction using the third image data and certain second image data representing the regions surrounding each histologic feature.

In some examples, the process 500 may further include outputting the third image data for validation by an authorized user, and updating the third image data based at least in part on updates provided by the authorized user. In this example, generating the clinical prediction comprises generating the clinical prediction using the updated third image data.

At block 510, the process 500 includes the computer system providing information associated with the clinical prediction for presentation, which may include presentation at a display of an electronic device such as the computer system or a different computer system.

In some examples, providing the information associated with the clinical prediction may include outputting an indication of the target clinical diagnosis, a score corresponding to the clinical prediction, and at least a portion of the third image data that supports the clinical prediction.

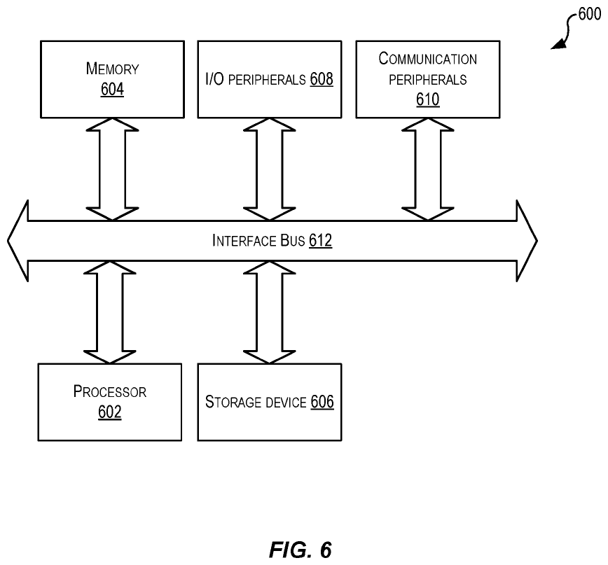

FIG. 6 illustrates examples of components of a computer system 600, according to at least one example. The computer system 600 may be a single computer such as a user's computing device and/or can represent a distributed computing system such as one or more server computing devices. The computer system 600 is an example of the local computer system 204 and the remote computer system 206.

The computer system 600 may include at least a processor 602, a memory 604, a storage device 606, input/output peripherals (I/O) 608, communication peripherals 610, and an interface bus 612. The interface bus 612 is configured to communicate, transmit, and transfer data, controls, and commands among the various components of the computer system 600. The memory 604 and the storage device 606 include computer-readable storage media, such as random-access memory (RAM), Read ROM, electrically erasable programmable read-only memory (EEPROM), hard drives, CD-ROMs, optical storage devices, magnetic storage devices, electronic non-volatile computer storage, for example Flash® memory, and other tangible storage media. Any of such computer-readable storage media can be configured to store instructions or program codes embodying aspects of the disclosure. The memory 604 and the storage device 606 also include computer-readable signal media. A computer-readable signal medium includes a propagated data signal with computer-readable program code embodied therein. Such a propagated signal takes any of a variety of forms including, but not limited to, electromagnetic, optical, or any combination thereof. A computer-readable signal medium includes any computer-readable medium that is not a computer-readable storage medium and that can communicate, propagate, or transport a program for use in connection with the computer system 600.

Further, the memory 604 includes an operating system, programs, and applications. The processor 602 is configured to execute the stored instructions and includes, for example, a logical processing unit, a microprocessor, a digital signal processor, and other processors. The memory 604 and/or the processor 602 can be virtualized and can be hosted within another computing system of, for example, a cloud network or a data center. The I/O peripherals 608 include user interfaces, such as a keyboard, screen (e.g., a touch screen), microphone, speaker, other input/output devices, and computing components, such as graphical processing units, serial ports, parallel ports, universal serial buses, and other input/output peripherals. The I/O peripherals 608 are connected to the processor 602 through any of the ports coupled to the interface bus 612. The communication peripherals 610 are configured to facilitate communication between the computer system 600 and other computing devices over a communications network and include, for example, a network interface controller, modem, wireless and wired interface cards, antenna, and other communication peripherals.

In the following, further clauses are described to facilitate the understanding of the present disclosure.

Clause 1. In this clause, there is provided a computer-implemented method, comprising:
receiving first image data corresponding to a tissue sample, wherein the first image data represents an autofluorescence image;
generating second image data from the first image data by applying at least one virtual stain to the first image data, wherein the at least one virtual stain is selected based on a target clinical diagnosis;
generating, by a predictive modeling suite, third image data from the second image data by identifying a plurality of histologic features present in the second image data in accordance with the target clinical diagnosis;
generating, by the predictive modeling suite and using the third image data, a clinical prediction relating to the target clinical diagnosis; and
providing information associated with the clinical prediction for presentation.

Clause 2. The computer-implemented method of clause 1, wherein generating the third image data from the second image data comprises generating the third image data by a first stage of the predictive modeling suite, and wherein generating the clinical prediction comprises generating the clinical prediction by a second stage of the predictive modeling suite.

Clause 3. The computer-implemented method of clause 2, wherein the first stage of the predictive modeling suite comprises a plurality of predictive models, and the second stage of the predictive modeling suite comprises a plurality of scoring models corresponding to the plurality of predictive models.

Clause 4. The computer-implemented method of clause 3, wherein the target clinical diagnosis is associated with a plurality of diagnosis symptoms, and wherein each predictive model of the plurality of predictive models and each scoring model of the plurality of scoring models is configured according to a diagnosis symptom of the plurality of diagnosis symptoms.

Clause 5. The computer-implemented method of clause 1, wherein the target clinical diagnosis is associated with a plurality of diagnosis symptoms.

Clause 6. The computer-implemented method of clause 5, wherein generating the clinical prediction comprises:
generating a clinical score for each diagnosis symptom of the plurality of diagnosis symptoms based on a respective histologic feature of the plurality of histologic features;
generating a composite score for the target clinical diagnosis based on the clinical score of each respective diagnosis symptom; and
generating the clinical prediction using the composite score.

Clause 7. The computer-implemented method of clause 5, wherein each diagnosis symptom of the plurality of diagnosis symptoms is associated with a diagnostic rule set, and wherein generating the clinical prediction comprises generating the clinical prediction in accordance with the diagnostic rule set.

Clause 8. The computer-implemented method of clause 1, wherein the second image data corresponds to a second image that is human-interpretable.

Clause 9. The computer-implemented method of clause 1, wherein the third image data corresponds to a set of heatmaps, wherein each heatmap of the set of heatmaps represents a histologic feature of the plurality of histologic features.

Clause 10. The computer-implemented method of clause 9, further comprising outputting the set of heatmaps as a set of annotation overlays, wherein each annotation overlay of the set of annotation overlays is selectively viewable with respect to the second image data.

Clause 11. The computer-implemented method of clause 1, wherein the third image data corresponds to annotations of each histologic feature of the plurality of histologic features, wherein the third image data is associated with a region of a second image represented by the second image data, and wherein generating the clinical prediction comprises generating the clinical prediction using the third image data and certain second image data representing the regions surrounding each histologic feature.

Clause 12. The computer-implemented method of clause 1, further comprising:
outputting the third image data for validation by an authorized user; and
updating the third image data based at least in part on updates provided by the authorized user, and wherein generating the clinical prediction comprises generating the clinical prediction using the updated third image data.

Clause 13. The computer-implemented method of clause 1, wherein providing the information associated with the clinical prediction comprises outputting an indication of the target clinical diagnosis, a score corresponding to the clinical prediction, and at least a portion of the third image data that supports the clinical prediction.

Clause 14. The computer-implemented method of clause 1, wherein the first image data corresponding to the tissue sample represents a plurality of color channels.

Clause 15. The computer-implemented method of clause 14, further comprising, prior to receiving the first image data, causing a microscope system to capture the first image data.

Clause 16. The computer-implemented method of clause 15, wherein the microscope system comprises a hyperspectral microscope and an imaging system.

Clause 17. The computer-implemented method of clause 15, wherein the microscope system captures the first image data using a plurality of different light frequencies and a plurality of different emission frequencies to produce a plurality of channels.

Clause 18. The computer-implemented method of clause 1, wherein the at least one virtual stain emulates a true tissue stain.

Clause 19. The computer-implemented method of clause 1, wherein receiving the first image data comprises receiving the first image data from a remote computer system, and wherein providing the information associated with the clinical prediction for presentation comprises providing the information for presentation at a display of the remote computer system.

Clause 20. A computer system, comprising:
a memory configured to store computer-executable instructions; and
a processor configured to access the memory and execute the computer-executable instructions to at least perform the method of any of clauses 1-19.

Clause 21. One or more non-transitory computer-readable media comprising computer-executable instructions that, when executed by one or more processors of a computer system, cause the computer system to perform the method of any of clauses 1-19.

Clause 22. A system, comprising:
a microscope system configured to capture a first set of images of a tissue sample;
a virtual stainer model configured to:
   receive the first set of images from the microscope system; and
   convert the first set of images into a second set of images based at least in part on color information associated with a target clinical diagnosis, wherein each image in the second set of images comprises a virtual stain;
a stage one predictive model configured to:
   receive, as input, the second set of images; and
   output a third set of images by converting the second set of images into the third set of images based at least in part on histologic feature information associated with the target clinical diagnosis, wherein each image in the third set of images comprises one or more histologic annotations corresponding to the target clinical diagnosis; and
a stage two predictive model configured to:
   receive, as input, the third set of images; and
   output a prediction corresponding to the target clinical diagnosis based at least in part on the histologic feature information.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art. Indeed, the methods and systems described herein may be embodied in a variety of other forms; furthermore, various omissions, substitutions, and changes in the form of the methods and systems described herein may be made without departing from the spirit of the present disclosure. The accompanying claims and their equivalents are intended to cover such forms or modifications as would fall within the scope and spirit of the present disclosure.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provide a result conditioned on one or more inputs. Suitable computing devices include multipurpose microprocessor-based computing systems accessing stored software that programs or configures the computing system from a general purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Embodiments of the methods disclosed herein may be performed in the operation of such computing devices. The order of the blocks presented in the examples above can be varied—for example, blocks can be re-ordered, combined, and/or broken into sub-blocks. Certain blocks or processes can be performed in parallel.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without author input or prompting, whether these features, elements and/or steps are included or are to be performed in any particular example.

Disjunctive language such as the phrase “at least one of X, Y, or Z,” unless specifically stated otherwise, is otherwise understood within the context as used in general to present that an item, term, etc., may be either X, Y, or Z, or any combination thereof (e.g., X, Y, and/or Z). Thus, such disjunctive language is not generally intended to, and should not, imply that certain examples require at least one of X, at least one of Y, or at least one of Z to each be present.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and all three of A and B and C.

The use of the terms “a” and “an” and “the” and similar referents in the context of describing the disclosed examples (especially in the context of the following claims) are to be construed to cover both the singular and the plural, unless otherwise indicated herein or clearly contradicted by context. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another, or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

All references, including publications, patent applications, and patents, cited herein are hereby incorporated by reference to the same extent as if each reference were individually and specifically indicated to be incorporated by reference and were set forth in its entirety herein.

1. A computer-implemented method, comprising:
receiving first image data corresponding to a tissue sample, wherein the first image data represents an autofluorescence image;
generating second image data from the first image data by applying at least one virtual stain to the first image data, wherein the at least one virtual stain is selected based on a target clinical diagnosis;
generating, by a predictive modeling suite, third image data from the second image data by identifying a plurality of histologic features present in the second image data in accordance with the target clinical diagnosis;
generating, by the predictive modeling suite and using the third image data, a clinical prediction relating to the target clinical diagnosis; and
providing information associated with the clinical prediction for presentation.

2. The computer-implemented method of claim 1, wherein generating the third image data from the second image data comprises generating the third image data by a first stage of the predictive modeling suite, and wherein generating the clinical prediction comprises generating the clinical prediction by a second stage of the predictive modeling suite.

3. The computer-implemented method of claim 2, wherein the first stage of the predictive modeling suite comprises a plurality of predictive models, and the second stage of the predictive modeling suite comprises a plurality of scoring models corresponding to the plurality of predictive models.

4. The computer-implemented method of claim 3, wherein the target clinical diagnosis is associated with a plurality of diagnosis symptoms, and wherein each predictive model of the plurality of predictive models and each scoring model of the plurality of scoring models is configured according to a diagnosis symptom of the plurality of diagnosis symptoms.

5. The computer-implemented method of claim 1, wherein the target clinical diagnosis is associated with a plurality of diagnosis symptoms.

6. The computer-implemented method of claim 5, wherein generating the clinical prediction comprises:
generating a clinical score for each diagnosis symptom of the plurality of diagnosis symptoms based on a respective histologic feature of the plurality of histologic features;
generating a composite score for the target clinical diagnosis based on the clinical score of each respective diagnosis symptom; and
generating the clinical prediction using the composite score.

7. The computer-implemented method of claim 5, wherein each diagnosis symptom of the plurality of diagnosis symptoms is associated with a diagnostic rule set, and wherein generating the clinical prediction comprises generating the clinical prediction in accordance with the diagnostic rule set.

8. The computer-implemented method of claim 1, wherein the second image data corresponds to a second image that is human-interpretable.

9. The computer-implemented method of claim 1, wherein the third image data corresponds to a set of heatmaps, wherein each heatmap of the set of heatmaps represents a histologic feature of the plurality of histologic features.

10. The computer-implemented method of claim 9, further comprising outputting the set of heatmaps as a set of annotation overlays, wherein each annotation overlay of the set of annotation overlays is selectively viewable with respect to the second image data.

11. The computer-implemented method of claim 1, wherein the third image data corresponds to annotations of each histologic feature of the plurality of histologic features, wherein the third image data is associated with a region of a second image represented by the second image data, and wherein generating the clinical prediction comprises generating the clinical prediction using the third image data and certain second image data representing the regions surrounding each histologic feature.

12. The computer-implemented method of claim 1, further comprising:
outputting the third image data for validation by an authorized user; and
updating the third image data based at least in part on updates provided by the authorized user, and wherein generating the clinical prediction comprises generating the clinical prediction using the updated third image data.

13. The computer-implemented method of claim 1, wherein providing the information associated with the clinical prediction comprises outputting an indication of the target clinical diagnosis, a score corresponding to the clinical prediction, and at least a portion of the third image data that supports the clinical prediction.

14. The computer-implemented method of claim 1, wherein the first image data corresponding to the tissue sample represents a plurality of color channels.

15. The computer-implemented method of claim 14, further comprising, prior to receiving the first image data, causing a microscope system to capture the first image data.

16. The computer-implemented method of claim 15, wherein the microscope system comprises a hyperspectral microscope and an imaging system.

17. The computer-implemented method of claim 15, wherein the microscope system captures the first image data using a plurality of different light frequencies and a plurality of different emission frequencies to produce a plurality of channels.

18. The computer-implemented method of claim 1, wherein the at least one virtual stain emulates a true tissue stain.

19. (canceled)

20. A computer system, comprising:
a memory configured to store computer-executable instructions; and
a processor configured to access the memory and execute the computer-executable instructions to:
receive first image data corresponding to a tissue sample, wherein the first image data represents an autofluorescence image;
generate second image data from the first image data by applying at least one virtual stain to the first image data, wherein the at least one virtual stain is selected based on a target clinical diagnosis;
generate, by a predictive modeling suite, third image data from the second image data by identifying a plurality of histologic features present in the second image data in accordance with the target clinical diagnosis;
generate, by the predictive modeling suite and using the third image data, a clinical prediction relating to the target clinical diagnosis; and
provide information associated with the clinical prediction for presentation.

21. One or more non-transitory computer-readable media comprising computer-executable instructions that, when executed by one or more processors of a computer system, cause the computer system to:
receive first image data corresponding to a tissue sample, wherein the first image data represents an autofluorescence image;
generate second image data from the first image data by applying at least one virtual stain to the first image data, wherein the at least one virtual stain is selected based on a target clinical diagnosis;
generate, by a predictive modeling suite, third image data from the second image data by identifying a plurality of histologic features present in the second image data in accordance with the target clinical diagnosis;
generate, by the predictive modeling suite and using the third image data, a clinical prediction relating to the target clinical diagnosis; and
provide information associated with the clinical prediction for presentation.

22. (canceled)
